(19) United States
(12) Patent Application Publication
(10) Pub. No.: US 2026/0060583 A1
(43) Pub. Date: Mar. 5, 2026

(54) METHOD, APPARATUS AND PROGRAM FOR MEASURING MUSCLE STRENGTH USING EXERCISE ASSISTANT ROBOT

(71) Applicant: UIF (University Industry Foundation), Yonsei University, Seoul (KR)

(72) Inventor: Jinyoung PARK, Seoul (KR)

(73) Assignee: UIF (University Industry Foundation), Yonsei University, Seoul (KR)

(21) Appl. No.: 19/312,449

(22) Filed: Aug. 28, 2025

(30) Foreign Application Priority Data
Sep. 2, 2024 (KR) 10-2024-0118876

(51) Int. Cl. A61B 5/22; A63B 21/00; A63B 21/002; A63B 24/00; B25J 11/00; G16H 40/63

(52) U.S. Cl. CPC A61B 5/224; A63B 21/00181; A63B 21/0023; A63B 24/0062; B25J 11/008; G16H 40/63; A63B 2220/833

(57) ABSTRACT

Disclosed is a method, apparatus, and program for measuring muscle strength by controlling an exercise assistance robot with customized settings for each user to measure the user's muscle strength, and may include receiving input information including muscle group information of a muscle strength measurement target of a user and body information of the user; setting automatically a position and shape of the exercise assistance robot for muscle strength measurement based on the muscle group information and the body information; setting automatically a movement path of the muscle strength measurement target based on the body information of the user; measuring the muscle strength for the muscle strength measurement target by controlling the exercise assistance robot based on the movement path of the muscle strength measurement target; and generating muscle strength measurement result information for the muscle strength measurement target.

### CROSS-REFERENCE TO RELATED APPLICATIONS

A claim for priority under 35 U.S.C. § 119 is made to Korean Patent Application No. 10-2024-0118876 filed on Sep. 2, 2024 in the Korean Intellectual Property Office, the entire contents of which are hereby incorporated by reference.

### BACKGROUND

### 1. Technical Field

The present disclosure relates to a method for measuring muscle strength, and more specifically, to a method, apparatus, and program for measuring muscle strength of a patient by controlling an exercise assistance robot with a customized setting for each patient.

### 2. Description of Related Art

With the advent of an aging society, the number of stroke patients is increasing, as is the number of spinal cord injuries from traffic accidents. Furthermore, the number of patients with impaired hand movement due to various diseases, the brain damage such as stroke, traumatic brain injury, cerebral palsy, or the nervous system damage caused by spinal cord injury, is also increasing.

Various rehabilitation medical devices have been developed to restore lost musculoskeletal functions in these patients, facilitating medical rehabilitation and smooth social activities for the elderly, the elderly, and the disabled.

The existing ‘isokinetic exercise assessment equipment’ can measure muscle strength and endurance of large joints with rotational angles. However, the following limitations limit its widespread use:
1) Medical intervention is essential throughout the entire process of measuring and storing individual user joint movement data. The user's (patient's) body parts that includes the joints need be fixed to the equipment. Each time, the equipment needs to be manually adjusted to ensure a safe angle, considering the user's posture and limb length. This process is time-consuming and physically demanding for medical staff, but due to low reimbursement for the procedure, not many medical institutions actually purchase and use the equipment.
2) Furthermore, when the same user repeatedly measures the same joint movements or exercises on different days, the equipment's hardware settings cannot be remembered, forcing medical staff to repeat the time and effort invested in the first measurement for the second time. This inefficient situation has not been improved.
3) Furthermore, when a user undergoes muscle strength assessments for various joint movements, replacing the accessory modules for measurement also requires significant time and physical effort. For example, to assess shoulder flexion and then hip extension, a shoulder joint measurement module weighing approximately 5 to 6 kg needs to be fixed to the equipment, then removed after the test, and a hip joint measurement module weighing approximately 6 to 7 kg needs to be installed. This process takes approximately 15 minutes and places a significant physical burden on the evaluator.

Therefore, there is a need to develop a muscle strength measurement method that can measure muscle strength with customized settings for each user, utilizing an automated robot.

### SUMMARY

The present disclosure is to provide a method, apparatus, and program for measuring a muscle strength of a user and adjusting an exercise intensity with customized setting for each user using an exercise assistance robot.

Technical problems of the inventive concept are not limited to the technical problems mentioned above, and other technical problems not mentioned will be clearly understood by those skilled in the art from the following description.

In an aspect of the present disclosure, a method for measuring muscle strength using an exercise assistance robot may include receiving input information including muscle group information of a muscle strength measurement target of a user and body information of the user; setting automatically a position and shape of the exercise assistance robot for muscle strength measurement based on the muscle group information and the body information; setting automatically a movement path of the muscle strength measurement target based on the body information of the user; measuring the muscle strength for the muscle strength measurement target by controlling the exercise assistance robot based on the movement path of the muscle strength measurement target; and generating muscle strength measurement result information for the muscle strength measurement target.

In an embodiment, the muscle group information may include joints of upper and lower extremities of the user's body and a movement direction of the joints.

In an embodiment, the body information of the user may include a length between the joints of the upper and lower extremities of the user's body.

In an embodiment, setting automatically the position and shape of the exercise assistance robot may include controlling the exercise assistance robot to move to the position of the muscle strength measurement target of the user based on the muscle group information of the user, determining a specific position of the muscle strength measurement target based on the body information of the user, and controlling the exercise assistance robot to contact the specific position of the determined muscle group, thereby automatically setting the exercise assistance robot.

In an embodiment, the specific position of the muscle group of the muscle strength measurement target is determined based on the length between joints corresponding to the muscle group of the muscle strength measurement target of the user.

In an embodiment, setting automatically the position and shape of the exercise assistance robot may include automatically storing a setting value of the exercise assistance robot corresponding to the muscle group of the muscle strength measurement target for each user upon completion of an initial setting of the exercise assistance robot.

In an embodiment, setting automatically the movement path of the muscle strength measurement target may include: based on the exercise assistance robot being automatically set to a specific position of the muscle strength measurement target, predicting a rotation radius of the muscle strength measurement target based on length information between the joints corresponding to the muscle strength measurement target among the body information of the user, and automatically setting the movement path of the muscle strength measurement target based on the predicted rotation radius.

In an embodiment, measuring the muscle strength for the muscle strength measurement target may include controlling the exercise assistance robot based on the movement path of the muscle strength measurement target to measure a maximum muscle strength of each joint movement through an isometric exercise.

In an embodiment, generating the muscle strength measurement result information may include generate result information visualizing the muscle strength measurement result for the muscle strength measurement target.

In another aspect of the present disclosure, a computer program stored on a computer-readable storage medium, the computer program, when executed on one or more processors, causes the computer program to perform the following operations for measuring a muscle strength of a user using an exercise assistance robot, the operations comprising: receiving input information including muscle group information of a muscle strength measurement target of a user and body information of the user; setting automatically a position and shape of the exercise assistance robot for muscle strength measurement based on the muscle group information and the body information; setting automatically a movement path of the muscle strength measurement target based on the body information of the user; measuring the muscle strength for the muscle strength measurement target by controlling the exercise assistance robot based on the movement path of the muscle strength measurement target; and generating muscle strength measurement result information for the muscle strength measurement target.

In still another aspect of the present disclosure, a computing device for providing a muscle strength measurement method using an exercise assistance robot may include a processor including one or more cores; and a memory, wherein the processor is configured to: receive input information including muscle group information of a muscle strength measurement target of a user and body information of the user; set automatically a position and shape of the exercise assistance robot for muscle strength measurement based on the muscle group information and the body information; set automatically a movement path of the muscle strength measurement target based on the body information of the user; measure the muscle strength for the muscle strength measurement target by controlling the exercise assistance robot based on the movement path of the muscle strength measurement target; and generate muscle strength measurement result information for the muscle strength measurement target.

A computer program providing a muscle strength measurement method according to another embodiment of the present disclosure is coupled with a computer as hardware and stored on a medium to perform any one of the above-described methods.

In addition, other methods for implementing the present disclosure, other systems, and computer-readable recording media recording a computer program for executing the above-described method may be further provided.

### BRIEF DESCRIPTION OF THE FIGURES

FIG. 1 is a block diagram illustrating a computing device performing operations for providing a muscle strength measurement method according to an embodiment of the present disclosure.

FIGS. 2 to 5 are diagrams illustrating a muscle strength measurement process using an exercise assistance robot according to an embodiment of the present disclosure.

FIGS. 6 to 10 are diagrams illustrating result information visualizing muscle strength measurement according to one embodiment of the present disclosure.

FIG. 11 is a flowchart illustrating a muscle strength measurement method according to one embodiment of the present disclosure.

### DETAILED DESCRIPTION

The advantages and features of the present disclosure, and methods for achieving them, will become clear with reference to the embodiments described in detail below along with the accompanying drawings. However, the present disclosure is not limited to the embodiments disclosed below and may be implemented in various different forms. The present embodiments are merely provided to ensure that the present disclosure is complete, and provided to fully convey the scope of the present disclosure to those skilled in the art to which the present disclosure pertains. The present disclosure is defined only by the scope of the claims.

The terminology used herein is for the purpose of describing embodiments and is not intended to limit the disclosure. As used herein, singular forms also include plural forms, unless specifically stated otherwise in the context. As used in the specification, “comprises” and/or “comprising” does not exclude the presence or addition of one or more other elements in addition to the mentioned elements. Throughout the specification, the same reference numerals refer to the same elements, and “and/or” includes each and every combination of the elements mentioned. Although “first”, “second”, etc. are used to describe various elements, it is to be understood that these elements are not limited by these terms. These terms are merely used to distinguish one element from another. Accordingly, it should be understood that a first element mentioned below may also be a second element within the technical scope of the present invention.

Unless otherwise defined, all terms (including technical and scientific terms) used in this specification may be used with meanings commonly understood by those skilled in the art to which this disclosure pertains. Additionally, terms defined in commonly used dictionaries are not to be interpreted ideally or excessively unless clearly specifically defined.

Hereinafter, the embodiments of the present disclosure will be described in detail with reference to the attached drawings.

Prior to the description, the meanings of terms used in this specification are briefly explained. However, since the description of terms is intended to aid understanding of this specification, it should be noted that they are not used in a meaning limiting the technical spirit of the present disclosure unless explicitly stated to limit the present disclosure.

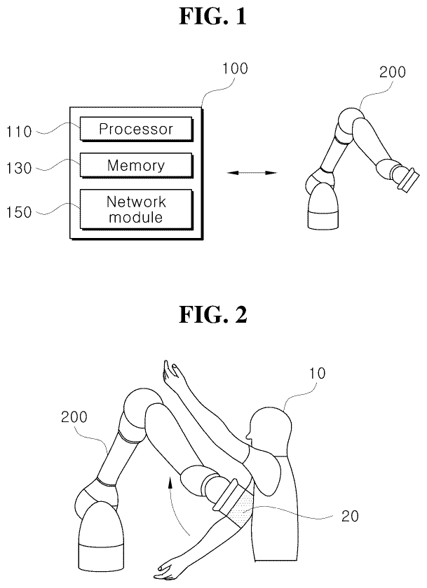

FIG. 1 is a block diagram illustrating a computing device performing operations for providing a muscle strength measurement method according to an embodiment of the present disclosure.

The configuration of a computing device 100 illustrated in FIG. 1 is merely a simplified example. In one embodiment of the present disclosure, the computing device 100 may include other components for implementing the computing environment of the computing device 100, and only some of the disclosed components may constitute the computing device 100.

The computing device 100 may include a processor 110, a memory 130, and a network module 150.

In the present disclosure, the processor 110 may receive input information including muscle group information of a muscle strength measurement target of a user and body information of the user, set automatically a position and shape of an exercise assistance robot 200 for muscle strength measurement based on the muscle group information and the body information, in addition, set automatically a rotation axes of the exercise assistance robot 200 to a specific position of the muscle strength measurement target, set automatically a movement path of the muscle strength measurement target based on the body information of the user, measure the muscle strength for the muscle strength measurement target by controlling the exercise assistance robot 200 based on the movement path of the muscle strength measurement target, and generate muscle strength measurement result information for the muscle strength measurement target.

Here, the muscle strength measurement target information of the user may include muscle group information, and joints of the upper and lower extremities of the user and movement directions of the corresponding joints. For example, the muscle group information may include information for shoulder joint flexors.

For example, the joint of the upper limb may include at least one of the shoulder joint, the elbow joint, or the wrist joint, the joint of the lower limb may include at least one of the knee joint, the hip joint, or the ankle joint, and the movement direction of the joint may include at least one of flexion, extension, abduction, adduction, external rotation, or internal rotation.

For example, the movement direction of the shoulder joint may include at least one of flexion, abduction, adduction, external rotation, or internal rotation; the movement direction of the elbow joint may include at least one of flexion or extension; the movement direction of the wrist joint may include at least one of flexion or extension; the movement direction of the hip joint may include at least one of flexion, extension, or abduction; the movement direction of the knee joint may include flexion or extension; and the movement direction of the ankle joint may include at least one of dorsiflexion or plantar flexion, but this is merely an embodiment and is not limited thereto.

In addition, the body information of the user may include a length between joints of the upper or lower limbs of the user's body.

For example, the body information of the user may include at least one of the length between the shoulder joint and the elbow joint, the length between the elbow joint and the wrist joint, the length between the wrist joint and the metacarpal joint, the length between the hip joint and the knee joint, the length between the knee joint and the ankle joint, or the length between the ankle joint and the metatarsal joint.

In addition, when receiving a user input, in the case that a user input requesting a muscle strength measurement is received, the processor 110 may provide a muscle strength measurement information input window, and receive a user input including user information, information for the muscle strength measurement target of the user, and body information through the muscle strength measurement information input window.

For example, when receiving the user input requesting muscle strength measurement, the processor 110 may provide an initial screen including a muscle strength measurement start button when the power is turned on, and recognize a user input selecting the muscle strength measurement start button as the user input requesting muscle strength measurement.

As another example, when the processor 110 receives the user input requesting muscle strength measurement, the processor 110 may activate a camera when the power is turned on, and recognize a user gesture corresponding to the start of muscle strength measurement through the camera's captured image as the user input requesting muscle strength measurement in the case that the user gesture is acquired through the camera's captured image.

As another example, when the processor 110 receives the user input requesting muscle strength measurement, the processor 110 may activate a microphone when the power is turned on, and recognize a user voice command corresponding to the start of muscle strength measurement through the microphone's voice reception as a user input requesting muscle strength measurement.

In addition, the muscle strength measurement information input window may include a first input field for entering user information, a second input field for entering user muscle strength measurement target information, and a third input field for entering user body information.

Here, the first input field may include at least one of a user name input item, a user date of birth input item, a user gender input item, a user height input item, or a user weight input item, but this is merely an embodiment and is not limited thereto.

The second input field may include joint input items of the upper and lower extremities and movement direction input items of the corresponding joints.

As an example, the joint input items of the upper extremities may include at least one of a shoulder joint input item, an elbow joint input item, or a wrist joint input item, the joint input item of the lower extremities may include at least one of a knee joint input item, a hip joint input item, or an ankle joint input item, and the movement direction input item of the joint may include at least one of a flexion input item, an extension input item, an abduction input item, an adduction input item, an external rotation input item, or an internal rotation input item, but this is merely an embodiment and is not limited thereto.

The third input field may include at least one of a length input item between the shoulder joint and the elbow joint, a length input item between the elbow joint and the wrist joint, a length input item between the wrist joint and the carpometacarpal joint, a length input item between the hip joint and the knee joint, a length input item between the knee joint and the ankle joint, or a length input item between the ankle joint and the tarsometatarsal joint, but this is merely an embodiment and is not limited thereto.

In addition, when receiving the user input through the muscle measurement information input window, in the case that the user information is input, the processor 110 may determine whether previously stored muscle measurement target information and body information of the user exist based on the user information, and in the case that the previously stored muscle measurement target information and the body information of the user exist, the processor may deactivate the second and third input fields among the first input field for inputting the user information included in the muscle measurement information input window, the second input field for inputting the muscle measurement target information of the user, and the third input field for inputting the body information of the user.

Here, when the second and third input fields are deactivated, the processor 110 may generate and provide a message window inquiring whether to re-enter the muscle strength measurement target information and body information of the user.

For example, the message window may include a query message inquiring whether to re-enter the muscle strength measurement target information and body information of the user, a re-entry consent button for the query message, and a re-entry rejection button for the query message.

Furthermore, when generating and providing the message window, in the case that the processor 110 requests re-entry of the muscle strength measurement target information and body information of the user through the message window, the processor 110 may re-activate the deactivated second and third input fields.

In some cases, when generating and providing the message window, in the case that the processor 110 refuses re-entry of the muscle strength measurement target information and body information of the user through the message window, the processor 110 may maintain the deactivated second and third input fields in a deactivated state.

Next, when automatically setting the exercise assistance robot, the processor 110 may control the exercise assistance robot to move to the user's muscle strength measurement target position based on the muscle strength measurement target information of the user, determine a specific position of the muscle strength measurement target based on the body information of the user, and control the exercise assistance robot to contact the determined specific position of the muscle strength measurement target, thereby automatically setting the exercise assistance robot.

Here, the specific position of the muscle strength measurement target refers to a position recognized by a sensor of the exercise assistance robot, and may be determined based on the length between joints corresponding to the muscle strength measurement target of the user.

As an example, when determining the specific position of the muscle strength measurement target, in the case that the muscle strength measurement target of the user is the shoulder joint and the movement direction of the shoulder joint is flexion or abduction, the processor 110 may determine the central region between the user's shoulder joint and the elbow joint as the specific position based on the length between the user's shoulder joint and the elbow joint.

As another example, the processor 110 may determine the central region between the user's elbow joint and the wrist joint as the specific position based on the length between the user's elbow joint and the wrist joint when the user's muscle strength measurement target is the shoulder joint and the movement direction of the shoulder joint is external rotation.

As still another example, when determining the specific position of the muscle strength measurement target, the processor 110 may determine the central region between the user's elbow joint and the wrist joint as the specific position when the user's muscle strength measurement target is the knee joint and the movement direction of the knee joint is extension. Based on the length between the user's knee joint and ankle joint, the central area between the user's knee joint and ankle joint may be determined as the specific position.

As still another example, when determining the specific position of the muscle strength measurement target, in the case that the muscle strength measurement target of the user is the elbow joint and the movement direction of the elbow joint is flexion or extension, the processor 110 may determine the central area between the elbow joint and the wrist joint as the specific position while a portion of the user's upper limb is in contact with the table auxiliary device.

As still another example, when determining the specific position of the muscle strength measurement target, in the case that the muscle strength measurement target of the user is the wrist joint and the movement direction of the wrist joint is flexion, the processor 110 may determine the central area between the wrist joint and the finger joint as the specific position while a portion of the user's upper limb is in contact with the table auxiliary device.

As still another example, when determining the specific position of the muscle strength measurement target, in the case that the muscle strength measurement target of the user is the hip joint and the movement direction of the hip joint is any one of flexion, abduction, and extension, the processor 110 may determine the central region between the user's hip joint and the knee joint and the central region between the user's knee joint and the ankle joint as the specific position while the user is lying on the bed assistance device.

As still another example, when determining the specific position of the muscle strength measurement target, in the case that the muscle strength measurement target of the user is the ankle joint and the movement direction of the ankle joint is dorsiflexion or plantarflexion, the processor 110 may determine the central region between the ankle joint and the toe joint as the specific position while a part of the user's lower extremity is in contact with an assistance device including a chair or a footrest.

Next, when automatically setting the exercise assistance robot 200, the processor 110 may automatically store the setting value of the exercise assistance robot corresponding to the muscle strength measurement target for each user when the initial setting of the exercise assistance robot 200 is completed.

In addition, the processor 110 may automatically set the exercise assistance robot 200 based on the initial value for each user when re-measuring the muscle strength measurement target.

That is, when initially setting up the exercise assist robot for an individual user, the processor 110 may acquire information for the user, and check whether there is a setting value of the exercise assist robot 200 corresponding to the muscle strength measurement target of the user pre-stored in the memory 130 based on the user's information, and in the case that there is a setting value of the exercise assist robot 200 corresponding to the muscle strength measurement target of the user pre-stored in the memory 130, the processor may extract the setting value of the exercise assist robot 200 corresponding to the muscle strength measurement target of the user pre-stored, and automatically reset the exercise assist robot 200 based on the extracted setting value of the exercise assist robot 200.

Here, when automatically setting the exercise assistance robot 200, the processor 110 may generate and provide a message window inquiring whether to automatically set the exercise assistance robot 200 with the pre-stored setting value in the case that there is the pre-stored setting value.

As an example, the message window may include an inquiry message inquiring whether to automatically set the exercise assistance robot 200 with the pre-stored setting value, an agree button for the inquiry message, and a reject button for the inquiry message.

When generating and providing the message window, the processor 110 may automatically set the exercise assistance robot 200 with the pre-stored setting value in the case that a request for automatic setting is made through the message window with the pre-stored setting value of the exercise assistance robot 200.

In addition, when the processor 110 generates and provides a message window, in the case that the automatic setting with the pre-stored setting value of the exercise assistance robot 200 is rejected through the message window, the processor 110 may not perform automatic setting with the pre-stored setting value of the exercise assistance robot, but may newly set the setting value of the exercise assistance robot 200 based on the muscle strength measurement target information and body information of the user.

In addition, when automatically setting the movement path of the muscle strength measurement target, the processor 110 may automatically set the exercise assistance robot 200 to the specific position of the muscle strength measurement target, and may predict a rotation radius of the muscle strength measurement target based on the length information between joints corresponding to the muscle strength measurement target among the body information of the user, and may automatically set the movement path of the muscle strength measurement target in consideration of the predicted rotation radius.

Next, when measuring muscle strength of the muscle strength measurement target, the processor 110 may control the exercise assistance robot 200 based on the movement path of the muscle strength measurement target to measure the maximum muscle strength of the respective joint movement through isometric exercise.

Here, when measuring muscle strength of the muscle strength measurement target, the processor 110 may measure the muscle strength of the muscle strength measurement target through a process of gradually increasing the loading by evaluating in real time the user's muscle fatigue.

As an example, when evaluating in real time the user's muscle fatigue, the processor 110 may measure the duration of the isometric exercise of the muscle strength measurement target while setting the value to approximately 80 percent (%) of the maximum muscle strength, and may evaluate in real time user's muscle fatigue based on the measured duration.

In addition, when measuring muscle strength of the muscle strength measurement target, the processor 110 may measure the muscle strength of the muscle strength measurement target through a process of increasing or decreasing loading according to the user's muscle strength level. In some cases, when measuring the muscle strength of the muscle strength measurement target, the processor 110 may provide user guide information for muscle strength measurement on a display screen in the form of at least one of video, audio, or text before measuring the muscle strength of the muscle strength measurement target.

Next, when generating muscle strength measurement result information, the processor 110 may generate result information that visualizes the muscle strength measurement result of the muscle strength measurement target.

For example, the result information visualizing the muscle strength measurement result for the muscle strength measurement target may include at least one of the following: result information visualizing the displacement of the equilibrium point the point where the robot's resistance and the user's resistance reach the equilibrium point may be confirmed through the pressure sensor equipped in the computing device 100 according to the increase in loading as a graph; result information visualizing the user's maximum muscle strength value and muscle fatigue as a graph; result information visualizing the user's muscle strength evaluation information for each round of the evaluation as a graph; or result information visualizing the position coordinates of the exercise assistance robot for each round of the evaluation and the loading applied to the torque sensor accordingly as a graph. However, this is merely an embodiment and is not limited thereto.

Meanwhile, the exercise assistance robot 200 may measure the maximum muscle strength during isometric exercise through a force control function and a compliance control function.

Here, the exercise assistance robot 200 may track and follow a path such as a rotational motion or a hinge motion of a joint using six axes.

The exercise assistance robot 200 may include an end effector, such as a torque sensor, a pad, and an armband-type accessory, but this is merely an embodiment and is not limited thereto.

According to one embodiment of the present disclosure, the processor 110 may be constructed with one or more cores and may include a processor for data analysis, such as a central processing unit (CPU), a general purpose graphics processing unit (GPGPU), or a tensor processing unit (TPU) of a computing device. The processor 110 may read a computer program stored in the memory 130 and perform data processing according to one embodiment of the present disclosure. At least one of the CPU, GPGPU, or TPU of the processor 110 may process network algorithm learning. For example, the CPU and GPGPU may jointly process network algorithm learning and data classification using the network algorithm. Furthermore, in one embodiment of the present disclosure, processors of multiple computing devices may be used together to process data. Furthermore, a computer program executed on a computing device according to one embodiment of the present disclosure may be a CPU, GPGPU, or TPU executable program.

According to one embodiment of the present disclosure, the memory 130 may store a computer program for measuring muscle strength and providing muscle strength measurement results, and the stored computer program may be read and executed by the processor 120. The memory 130 may store any form of information generated or determined by the processor 110 and any form of information received by the network module 150.

According to one embodiment of the present disclosure, the memory 130 may include at least one type of storage medium among a flash memory type, a hard disk type, a multimedia card micro type, a card type memory (e.g., SD or XD memory, etc.), random access memory (RAM), static random access memory (SRAM), read-only memory (ROM), electrically erasable programmable read-only memory (EEPROM), programmable read-only memory (PROM), a magnetic memory, a magnetic disk, and an optical disk. The computing device 100 may also operate in relation to web storage that performs the storage function of the memory 130 on the Internet. The description of the above-described memory is merely an example and is not limited thereto.

The network module 150, according to one embodiment of the present disclosure, is connected to the exercise assistance robot 200 to transmit and receive control signals, muscle strength measurement result information, and the like to other computing devices, servers, and the like. In addition, the network module 150 may enable communication between multiple computing devices, thereby enabling operations for muscle strength measurement to be performed in a distributed manner on each of the multiple computing devices.

The network module 150 according to one embodiment of the present disclosure may operate based on any form of wired or wireless communication technology currently in use and implemented, such as short-range, long-range, wired, and wireless, and may also be used in other networks.

The computing device 100 according to the present disclosure may further include an output module and an input module.

The output module according to one embodiment of the present disclosure may display a user interface (UI) for providing the muscle strength measurement result. The output module may output any form of information generated or determined by the processor 110 and any form of information received by the network module 150.

In one embodiment of the present disclosure, the output module may include at least one of a liquid crystal display (LCD), a thin film transistor-liquid crystal display (TFT LCD), an organic light-emitting diode (OLED), a flexible display, and a three-dimensional (3D) display. Some of these display modules may be configured as transparent or light-transmitting so that the outside may be viewed through them. This may be referred to as a transparent display module, and a representative example of the transparent display module is a transparent OLED (TOLED).

According to one embodiment of the present disclosure, the input module may receive a user input. The input module may include keys and/or buttons on a user interface for receiving the user input, or physical keys and/or buttons. A computer program for controlling the display according to embodiments of the present disclosure may be executed based on the user input through the input module.

The input module according to embodiments of the present disclosure may receive a signal by detecting a user's button operation or touch input, or may receive a user's voice or motion through a camera or microphone and convert it into an input signal. For this purpose, the speech recognition technology or the motion recognition technology may be used.

The input module according to embodiments of the present disclosure may be implemented as an external input device connected to the computing device 100. For example, the input device may be at least one of a touchpad, a touch pen, a keyboard, or a mouse for receiving user input, but this is merely an example and is not limited thereto.

The input module according to one embodiment of the present disclosure may recognize a user's touch input. The input module according to one embodiment of the present disclosure may have the same configuration as the output module. The input module may be configured as a touch screen implemented to receive a user's selection input. The touch screen may use any one of a contact-type capacitive method, an infrared optical sensing method, a surface ultrasonic wave (SAW) method, a piezoelectric method, and a resistive film method. The detailed description of the touch screen described above is merely an example according to one embodiment of the present disclosure, and various touch screen panels may be employed in the computing device 100. The input module configured as a touch screen may include a touch sensor. The touch sensor may be configured to convert changes in pressure applied to a specific portion of the input module or in electrostatic capacity generated in a specific portion of the input module into an electrical input signal. The touch sensor may be configured to detect not only the position and area of the touch, but also the pressure at the time of the touch. When there is a touch input to the touch sensor, the corresponding signal(s) is sent to the touch controller. The touch controller may process the signal(s) and then transmit the corresponding data to the processor 110. As a result, the processor 110 may recognize which area of the input module has been touched, and the like.

In one embodiment of the present disclosure, the server may include other components for implementing a server environment. The server may include any type of device. The server may be a digital device equipped with a processor and memory and capable of computing, such as a laptop computer, a notebook computer, a desktop computer, a web pad, or a mobile phone.

A server (not shown) that performs an operation for providing a user interface displaying muscle strength measurement results to a user terminal according to one embodiment of the present disclosure may include a network module, a processor, and a memory.

The server may generate a user interface according to embodiments of the present disclosure. The server may be a computing system that provides information to a client (e.g., a user terminal) via a network. The server may transmit the generated user interface to the user terminal. In this case, the user terminal may be any type of computing device 100 that may access the server. The processor of the server may transmit the user interface to the user terminal via the network module. The server according to embodiments of the present disclosure may be, for example, a cloud server. The server may be a web server that processes services. The types of servers described above are merely examples and are not limiting.

As such, the present disclosure may easily assess maximum muscle strength for each joint movement through isometric exercise.

Furthermore, the present disclosure may store a setting value for each joint movement for each user.

Furthermore, the present disclosure utilizes an automated robot to assess a user's muscle strength, thereby reducing the time and labor required to set up heavy equipment and equipment accessories, as in the past. This reduces the physical burden on the evaluator and shortens the evaluation time.

Furthermore, the present disclosure may reduce the space required for evaluation.

Furthermore, the present disclosure may be moved by adding a mobile module, enabling evaluation in various positions, such as hospital rooms and intensive care units, rather than in a treatment room.

Furthermore, the present disclosure may generate new types of quantitative data in the healthcare and medical markets, enable seamless medical services connecting medical institutions, communities, and home exercise, enable planning of medical and commercial services through accumulated exercise data, and contribute to preventive medicine and economic market revitalization.

FIGS. 2 to 5 are diagrams illustrating a muscle strength measurement process using an exercise assistance robot according to an embodiment of the present disclosure.

As illustrated in FIGS. 2 to 5, the present disclosure may set automatically the exercise assistance robot 200 to a specific position 20 of the muscle strength measurement target based on muscle strength measurement target information and body information of a user 10, set automatically set a movement path of the muscle strength measurement target based on the body information of the user 10, and control the exercise assistance robot 200 based on the movement path of the muscle strength measurement target to measure muscle strength of the muscle strength measurement target.

Here, the present disclosure controls the exercise assistance robot 200 to move to the position of the muscle measurement target of the user 10 based on the muscle measurement target information of the user 10, determines the specific position 20 of the muscle measurement target based on the body information of the user 10, and controls the exercise assistance robot 200 to contact the determined specific position 20 of the muscle measurement target, thereby automatically setting the exercise assistance robot 200.

Here, the specific position 20 of the muscle measurement target may be determined based on the length between joints corresponding to the muscle measurement target of the user.

For example, as shown in FIG. 2, in the case that the muscle strength measurement target of the user 10 is the shoulder joint and the movement direction of the shoulder joint is flexion, the present disclosure may determine the front center area between the user's shoulder joint and the elbow joint as the specific position 20 based on the length between the shoulder joint and the elbow joint of the user 10, automatically set the movement path of the shoulder joint to the rotation radius of the plane where the forward flexion and extension of the user 10 occur, and control the exercise assistance robot 200 based on the movement path of the sensor to measure the muscle strength for flexion of the shoulder joint.

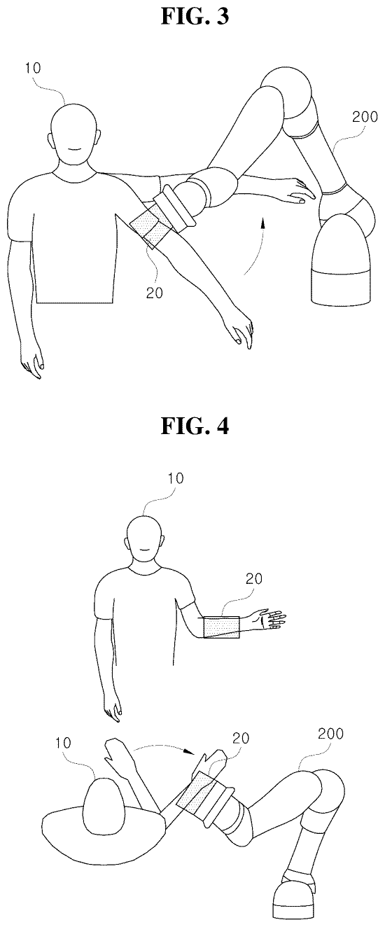

As another example, as shown in FIG. 3, in the case that the muscle strength measurement target of the user 10 is the shoulder joint and the movement direction of the shoulder joint is abduction, the present disclosure may determine the outer central region between the user's shoulder joint and the elbow joint as the specific position 20 based on the length between the shoulder joint and the elbow joint of the user 10, automatically set the movement path of the shoulder joint to the rotation radius of the plane where abduction and internal rotation of the shoulder joint of the user 10, and control the exercise assistance robot 200 based on the movement path of the sensor to measure the muscle strength for abduction of the shoulder joint.

As still another example, as shown in FIG. 4, in the case that the muscle strength measurement target of the user 10 is the shoulder joint and the movement direction of the shoulder joint is external rotation, the present disclosure may determine the central area between the elbow joint and the wrist joint of the user 10 as the specific position 20 based on the length between the elbow joint and the wrist joint, automatically set the movement path of the shoulder joint of the user 10 to the rotation radius of the plane where external rotation and internal rotation occur, and control the exercise assistance robot 200 based on the movement path of the sensor to measure the muscle strength for external rotation of the shoulder joint.

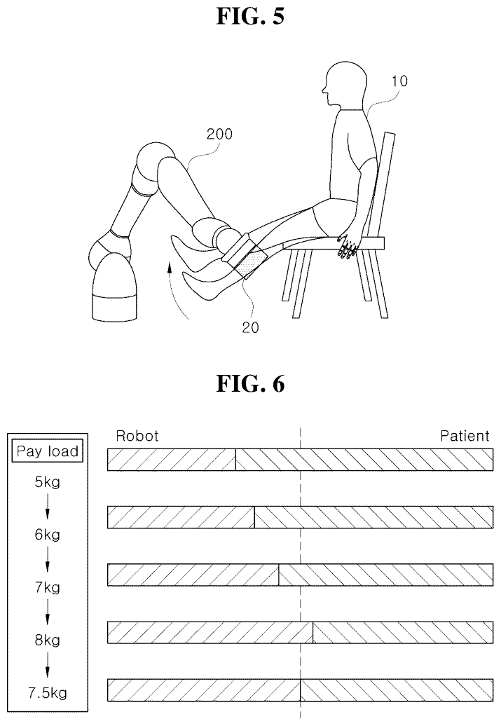

As still another example, as shown in FIG. 5, in the case that the muscle strength measurement target of the user 10 is the knee joint and the movement direction of the knee joint is extension, the present disclosure may determine the central area of the length between the knee joint and the ankle joint of the user 10 as the specific position 20, and automatically sets the movement path of the knee joint to the rotation radius of the plane where extension and flexion occur, and control the exercise assistance robot 200 based on the movement path of the sensor to measure the muscle strength for knee joint extension.

In addition, the present disclosure may automatically store the setting value of the exercise assistance robot 200 corresponding to the muscle strength measurement target for each user 10 once the initial setting of the exercise assistance robot 200 is completed.

In addition, the present disclosure may automatically set the exercise assistance robot 200 based on the pre-stored initial value for each user when re-measuring the muscle strength measurement target later.

For example, the present disclosure may acquire information for the user 10, check whether there is a setting value of the exercise assist robot 200 corresponding to the muscle strength measurement target of the user 10 pre-stored in the memory based on the user information, and in the case that there is a setting value of the exercise assist robot 200 corresponding to the muscle strength measurement target of the user 10 pre-stored in the memory, extract the setting value of the exercise assist robot 200 corresponding to the muscle strength measurement target of the user 10 pre-stored, and automatically reset the exercise assist robot 200 based on the extracted setting value of the exercise assist robot 200.

In addition, the present disclosure may extract length information between joints corresponding to the muscle strength measurement target from the body information of the user 10 when the exercise assistance robot 200 is automatically set to the specific position 20 of the muscle strength measurement target, predict the rotation radius of the muscle strength measurement target based on the extracted length information between the joints, and automatically set the movement path of the muscle strength measurement target by considering the predicted rotation radius.

In addition, the present disclosure may measure the maximum muscle strength of each joint movement through isometric exercise by controlling the exercise assistance robot 200 based on the movement path of the muscle strength measurement target.

The present disclosure may measure the muscle strength of the muscle strength measurement target by assessing the user's muscle fatigue in real time and gradually increasing the loading.

That is, the present disclosure may measure the duration of a muscle strength measurement target's isometric exercise while maintaining the strength at approximately 80 percent of the maximum strength, and may evaluate the user's muscle fatigue in real time based on the measured duration.

FIGS. 6 to 10 are diagrams illustrating result information visualizing muscle strength measurement according to one embodiment of the present disclosure.

As illustrated in FIGS. 6 to 10, the present disclosure may generate result information visualizing the muscle strength measurement results for the muscle strength measurement target.

For example, the result information visualizing the muscle strength measurement result for the muscle strength measurement target may include at least one of the following: result information visualizing the displacement of the equilibrium point according to the increase in loading as a graph; result information visualizing the user's maximum muscle strength value and muscle fatigue as a graph; result information visualizing the user's muscle strength evaluation information for each round of the user's muscle strength evaluation as a graph; or result information visualizing the position coordinates of the exercise assistance robot for each round of the user's muscle strength evaluation and the resulting loading on the torque sensor as a graph. However, this is merely an embodiment and is not limited thereto.

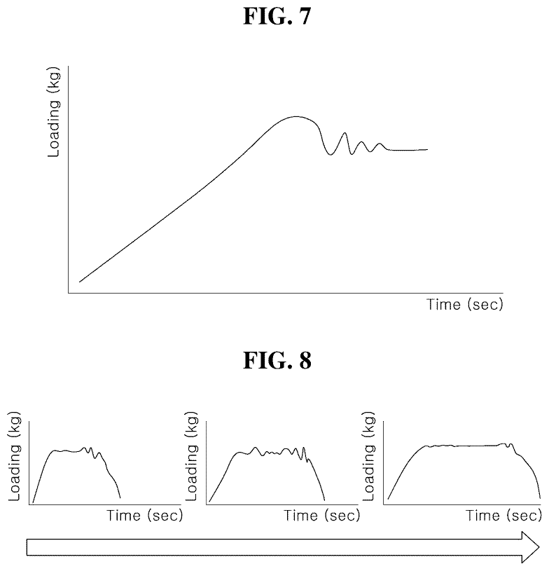

As shown in FIGS. 6 and 7, the present disclosure may provide the result information visualizing the measured muscle strength and the displacement of the equilibrium point according to the increase in loading as a graph through a process of gradually increasing the loading by evaluating the user's muscle strength and muscle fatigue in real time.

Furthermore, as illustrated in FIG. 8, the present disclosure may measure the duration of isometric exercise of a strength measurement target while maintaining the exercise at approximately 80 percent of the maximum strength, assess in real time whether the user is experiencing muscle fatigue based on the measured duration, and provide result information visualizing the user's maximum strength and muscle fatigue as a graph.

Furthermore, the present disclosure may store the setting value of the exercise assistance robot corresponding to the final step of the loading increase process for evaluating the user's muscle fatigue after completing the strength measurement of the strength measurement target, and may initiate the loading increase process for evaluating the user's muscle fatigue from the setting value of the exercise assistance robot corresponding to the final step during a subsequent strength measurement.

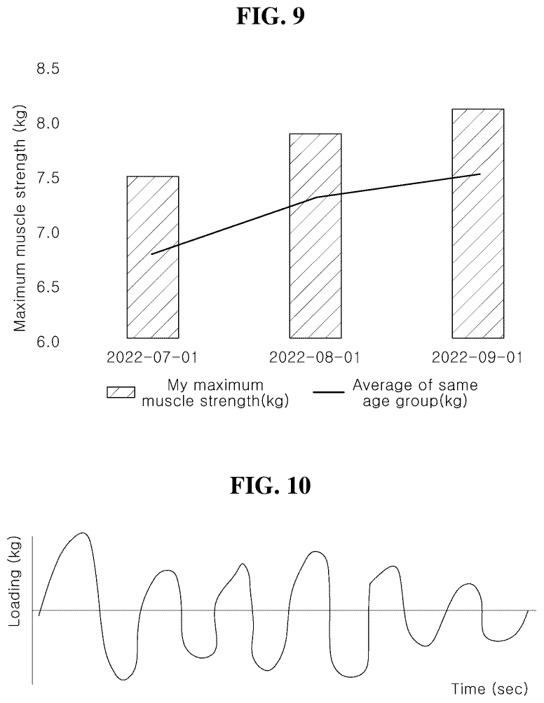

Here, as illustrated in FIG. 9, the present disclosure may provide the result information visualizing the user's muscle fatigue evaluation information for each muscle strength evaluation session as a graph.

Furthermore, as illustrated in FIG. 10, the present disclosure may measure muscle strength of the muscle strength measurement target by increasing or decreasing the loading according to the user's muscle strength level, and may provide the result information visualizing the position coordinates of the exercise assistance robot for each muscle strength evaluation session and the resulting loading on the torque sensor in a graph.

For example, in the present disclosure, in the case that the initial loading setting is approximately 7 kg, but the user's condition is good and muscle fatigue is not observed even after approximately 10 repetitions of approximately 7 kg, the loading may be increased by approximately 0.5 kg per 10 repetitions until muscle fatigue is observed.

Furthermore, the present disclosure may display the average loading value (kg) for each exercise session accumulated on the screen after the exercise is completed.

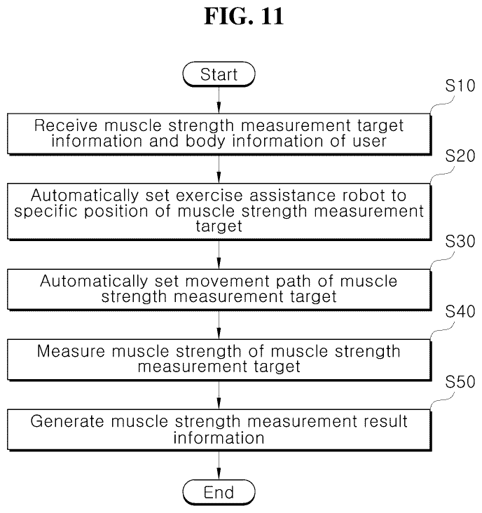

FIG. 11 is a flowchart illustrating a muscle strength measurement method according to one embodiment of the present disclosure.

As illustrated in FIG. 11, the present disclosure may receive a user input including muscle strength measurement target information and body information of the user (step S10).

Here, the user strength measurement target information may include joints of the upper and lower extremities of the user and the movement direction of the corresponding joints, and the body information of the user may include the length between joints of the user's upper or lower extremities.

Furthermore, the present disclosure may automatically set the exercise assistance robot to the specific position of the muscle strength measurement target based on the muscle strength measurement target information and body information of the user (step S20).

Here, the present disclosure may automatically set the exercise assistance robot by controlling the exercise assistance robot to move to the user's strength measurement target position based on the user's strength measurement target information, determining the specific position of the muscle measurement target based on the body information of the user, and controlling the exercise assistance robot to contact the determined specific position of the muscle measurement target.

The specific position of the muscle strength measurement target may be determined based on the length between joints corresponding to the user's muscle strength measurement target.

Subsequently, the present disclosure may automatically set the movement path of the muscle strength measurement target based on the body information of the user (step S30).

Here, the present disclosure may automatically set the exercise assistance robot to the specific position of the muscle strength measurement target, extract the length information between joints corresponding to the muscle strength measurement target from the body information of the user, predict the rotational radius of the muscle strength measurement target based on the extracted length information between joints, and automatically set the movement path of the muscle strength measurement target considering the predicted rotational radius.

Thereafter, the present disclosure may measure the muscle strength of the muscle strength measurement target by controlling the exercise assistance robot based on the movement path of the muscle strength measurement target (step S40).

Here, the present disclosure may measure the maximum muscle strength of each joint movement through isometric exercise by controlling the exercise assistance robot based on the movement path of the muscle strength measurement target.

As an example, the present disclosure may measure the muscle strength of the muscle strength measurement target by assessing the user's muscle fatigue in real time and gradually increasing the loading.

Furthermore, the present disclosure may generate muscle strength measurement result information for the muscle strength measurement target (step S50).

Here, the present disclosure may generate the result information visualizing the muscle strength measurement result for the muscle strength measurement target.

For example, the result information visualizing the muscle strength measurement result for the muscle strength measurement target may include at least one of: result information visualizing the displacement of the equilibrium point as a graph according to increased loading; result information visualizing the user's maximum muscle strength value and muscle fatigue as a graph; result information visualizing the user's muscle strength evaluation information for each round of the user's muscle strength evaluation for each round of the user's muscle strength evaluation for each round of the user's muscle strength evaluation for each round of the user's muscle strength evaluation for each round of the user's muscle strength evaluation; or result information visualizing the position coordinates of the exercise assistance robot and the resulting load on the torque sensor as a graph. However, this is merely an embodiment and is not limited thereto.

As such, the present disclosure may measure the user's muscle strength and adjust the exercise intensity using a user-specific customized setting using the exercise assistance robot.

Furthermore, the present disclosure may memorize the initial position value set during the initial evaluation and automatically set the initial position value during a re-evaluation, and automatically set the exercise path by inputting the length between joints, thereby enabling customized settings for each user.

Furthermore, the present disclosure may customize exercise intensity based on the results of an individual's maximum muscle strength assessment, and may gradually increase the load by assessing the user's muscle fatigue in real time during strength training, thereby customizing the strength training intensity for each user.

Furthermore, the present disclosure may easily evaluate the maximum muscle strength of each joint movement through isometric exercise.

Furthermore, the present disclosure may store setting values for each joint movement for each user.

Furthermore, the present disclosure may utilize an automated robot to evaluate a user's muscle strength, thereby reducing the time and labor required to set up heavy equipment and equipment accessories. This may reduce the physical burden on the evaluator and shorten the evaluation time.

Furthermore, the present disclosure may reduce the space required for evaluation.

In addition, the present disclosure may be evaluated in various places, such as a patient's room rather than a treatment room environment, because it may be moved by adding a mobile module.

In addition, the present disclosure may generate a new type of quantitative data in the healthcare and medical markets, and based on this, seamless medical services may be provided from medical institutions to the community to home exercise, and cumulative exercise may be performed. This data may enable the planning of medical and commercial services, contributing to preventive healthcare and economic market revitalization.

The method according to one embodiment of the present disclosure described above may be implemented as a program or application and stored on a medium to be executed in conjunction with a hardware server.

The program described above may include code coded in a computer language, such as C, C++, JAVA, or machine language, that may be read by the computer's processor CPU through the computer's device interface, so that the computer reads the program and executes the methods implemented as the program. This code may include functional code related to functions defining the functions necessary to execute the methods, and may include control code related to execution procedures necessary for the computer's processor to execute the functions according to a predetermined procedure. In addition, such code may further include memory reference-related code regarding the position address of the internal or external memory of the computer where additional information or media required for the computer's processor to execute the functions should be referenced. Furthermore, if the computer's processor requires communication with another remote computer or server to execute the functions, the code may further include communication-related code regarding how to communicate with another remote computer or server using the computer's communication module, and what information or media should be sent and received during communication.

The storage medium refers to a medium that stores data semi-permanently and may be read by a device, rather than a medium that stores data for a short period of time, such as a register, cache, or memory. Specifically, examples of the storage medium include, but are not limited to, ROM, RAM, CD-ROM, magnetic tape, floppy disk, and optical data storage devices. That is, the program may be stored on various recording media on various servers accessible by the computer or on various recording media on the user's computer. In addition, the media may be distributed across network-connected computer systems, so that computer-readable code may be stored in a distributed manner.

The steps of the method or algorithm described in connection with the embodiments of the present disclosure may be implemented directly in hardware, implemented as a software module executed by hardware, or implemented by a combination thereof. The software module may reside in a random access memory (RAM), a read only memory (ROM), an erasable programmable ROM (EPROM), an electrically erasable programmable ROM (EEPROM), a flash memory, a hard disk, a removable disk, a CD-ROM, or any other form of computer-readable recording medium well known in the art to which the present disclosure pertains.

While the embodiments of the present disclosure have been described with reference to the attached drawings, those skilled in the art will appreciate that the present disclosure can be implemented in other specific forms without altering the technical spirit or essential features thereof. Therefore, the embodiments described above should be understood to be illustrative in all respects and not restrictive.

What is claimed is:

1. A method for measuring muscle strength using an exercise assistance robot, comprising:
receiving input information including muscle group information of a muscle strength measurement target of a user and body information of the user;
setting automatically a position and shape of the exercise assistance robot for muscle strength measurement based on the muscle group information and the body information;
setting automatically a movement path of the muscle strength measurement target based on the body information of the user;
measuring the muscle strength for the muscle strength measurement target by controlling the exercise assistance robot based on the movement path of the muscle strength measurement target; and
generating muscle strength measurement result information for the muscle strength measurement target.

2. The method of claim 1, wherein the muscle group information includes joints of upper and lower extremities of the user's body and a movement direction of the joints.

3. The method of claim 1, wherein the body information of the user includes a length between the joints of the upper and lower extremities of the user's body.

4. The method of claim 1, wherein setting automatically the position and shape of the exercise assistance robot includes:
controlling the exercise assistance robot to move to the position of the muscle strength measurement target of the user based on the muscle group information of the user, determining a specific position of the muscle strength measurement target based on the body information of the user, and controlling the exercise assistance robot to contact the specific position of the determined muscle group, thereby automatically setting the exercise assistance robot.

5. The method of claim 4, wherein the specific position of the muscle group of the muscle strength measurement target is determined based on the length between joints corresponding to the muscle group of the muscle strength measurement target of the user.

6. The method of claim 1, wherein setting automatically the position and shape of the exercise assistance robot includes:
automatically storing a setting value of the exercise assistance robot corresponding to the muscle group of the muscle strength measurement target for each user upon completion of an initial setting of the exercise assistance robot.

7. The method of claim 1, wherein setting automatically the movement path of the muscle strength measurement target includes:
based on the exercise assistance robot being automatically set to a specific position of the muscle strength measurement target,
predicting a rotation radius of the muscle strength measurement target based on length information between the joints corresponding to the muscle strength measurement target among the body information of the user, and automatically setting the movement path of the muscle strength measurement target based on the predicted rotation radius.

8. The method of claim 1, wherein measuring the muscle strength for the muscle strength measurement target includes:
controlling the exercise assistance robot based on the movement path of the muscle strength measurement target to measure a maximum muscle strength of each joint movement through an isometric exercise.

9. A computer program stored on a computer-readable storage medium, the computer program, when executed on one or more processors, causes the computer program to perform the following operations for measuring a muscle strength of a user using an exercise assistance robot, the operations comprising:
receiving input information including muscle group information of a muscle strength measurement target of a user and body information of the user;
setting automatically a position and shape of the exercise assistance robot for muscle strength measurement based on the muscle group information and the body information;
setting automatically a movement path of the muscle strength measurement target based on the body information of the user;
measuring the muscle strength for the muscle strength measurement target by controlling the exercise assistance robot based on the movement path of the muscle strength measurement target; and
generating muscle strength measurement result information for the muscle strength measurement target.

10. A computing device for providing a muscle strength measurement method using an exercise assistance robot, comprising:
a processor including one or more cores; and
a memory,
wherein the processor is configured to:
receive input information including muscle group information of a muscle strength measurement target of a user and body information of the user;
set automatically a position and shape of the exercise assistance robot for muscle strength measurement based on the muscle group information and the body information;
set automatically a movement path of the muscle strength measurement target based on the body information of the user;
measure the muscle strength for the muscle strength measurement target by controlling the exercise assistance robot based on the movement path of the muscle strength measurement target; and
generate muscle strength measurement result information for the muscle strength measurement target.
